(19) United States
(12) Patent Application Publication
(10) Pub. No.: US 2023/0181927 A1
(43) Pub. Date: Jun. 15, 2023

(54) SUB-RETINAL PROSTHESIS USING MULTI-PHOTODIODE SENSING TECHNOLOGY

(71) Applicant: KOREA UNIVERSITY RESEARCH AND BUSINESS FOUNDATION, Seoul (KR)

(72) Inventors: Hyung-Min LEE, Seoul (KR); Seong-Woo KIM, Seoul (KR); Kyeongho EOM, Seoul (KR)

(73) Assignee: KOREA UNIVERSITY RESEARCH AND BUSINESS FOUNDATION, Seoul (KR)

(21) Appl. No.: 18/078,471

(22) Filed: Dec. 9, 2022

(30) Foreign Application Priority Data
Dec. 14, 2021 (KR) 10-2021-0178354

(51) Int. Cl. A61N 5/06; A61F 2/14

(52) U.S. Cl. CPC A61N 5/0622; A61N 5/0601; A61F 2/14; A61N 2005/0626; A61N 2005/0652

(57) ABSTRACT

Disclosed herein is a sub-retinal prosthesis using a multi-photodiode sensing technology. The sub-retinal prosthesis using a multi-photodiode sensing technology includes: a pixel array including a plurality of pixels of which k pixels are set as a unit group; and a digital controller controlling the pixel array to activate one of the k pixels as a stimulation electrode and activate the other k-1 pixels as return electrodes, wherein the unit group includes k photodiodes, a sensing circuit and a current driver, the sensing circuit outputs a stimulation parameter using currents each generated by the k photodiodes according to irradiation of light as an input, and the current driver outputs a stimulation current corresponding to the stimulation parameter.

### CROSS-REFERENCE TO RELATED APPLICATION

This application claims priority under 35 U.S.C. § 119(a) to Korean Patent Application No. 10-2021-0178354 filed on Dec. 14, 2021, in the Korean Intellectual Property Office, the disclosure of which is incorporated herein by reference in its entirety.

### BACKGROUND

### (a) Technical Field

The present disclosure relates to a sub-retinal prosthesis using a multi-photodiode sensing technology.

### (b) Background Art

Visual information entering an eyeball in the form of light from a normal visual path is converted into an electrical signal in the retina and delivered through the optic nerve.

Representative diseases of blindness include retinitis pigmentosa and age-related macula degeneration, which occurs because light stimulation is not recognized from a retinal stage due to the death of visual cells.

In Korea, 400,000 patients suffer from the age-related macula degeneration, and the number of patients suffering from the age-related macula degeneration is expected to increase to reach 570,000 in 2030. In a case of these patients, only a visual cell layer, which is an outer layer of the retina, is damaged and the remaining nerves remain relatively intact. Therefore, a treatment using a retinal prosthesis that may substitute for a function of the visual cell layer among various treatment methods is the most like to restore a visual function.

Argus II available from Second Sight Medical Products, Inc., in the United States is a first-generation retinal prosthesis that has been commercialized worldwide, is an epi-retinal system, and has a resolution of 60 pixels, and 350 patients have undergone surgery through Argus II. Alpha AMS available from Retinal Implant AG and a research team of Professor E. Zrenner at Tubingen University in Germany is another first-generation retinal prosthesis that has been commercialized, and is a sub-retinal prosthesis system.

However, currently, both companies have stopped producing and selling first-generation products.

Currently, Pixium Vision LLC in France and a research team of Professors T. Fujikado and M. Kamei of Osaka University in Japan are developing Prima retinal implant and a supra-retinal implantable STS device as second-generation products, respectively.

The Alpha IMS product developed in Germany has been mounted with 1500 pixels, but according to a clinical test result, there is a report that patients actually recognize a resolution of the Alpha IMS product as a resolution of about 60 pixels. This is because photodiode photosensor arrays respond to light to simultaneously stimulate bipolar cells.

FIGS. 1A and 1B are views for describing a current bleeding phenomenon in a retinal prosthesis according to the related art.

Referring to FIG. 1A, four return electrodes are positioned at end portions of the retinal prosthesis. Assuming that stimulation electrodes are activated in a ‘┐’ shape, as illustrated in FIG. 1 B, currents generated from the stimulation electrodes flow to the return electrodes to stimulate bipolar cells that should not respond.

As a result, a phenomenon in which edges of an image detected by patients are bled occurs.

FIG. 2 is a diagram illustrating a layout of stimulation/return electrodes for removing a current bleeding phenomenon.

FIG. 2 is disclosed in Korean Patent No. 10-1838150. Referring to FIG. 2, electrodes around stimulation electrodes are utilized as return electrodes using reconfigurable stimulation/return electrodes to remove a current bleeding phenomenon.

In order to use the reconfigurable stimulation/return electrodes, the respective electrodes operate as stimulation electrodes or return electrodes according to time.

As the reconfigurable stimulation/return electrodes are used, only one of pixels belonging to different groups operates as a stimulation electrode for each stimulation cycle. Electrodes other than the stimulation electrode become return electrodes, and sensing circuits (circuits serving to sense light in pixels) of pixels corresponding to the return electrodes do not operate. Photodiodes, which are the most important parts of the sensing circuits, are also left unused in the pixels corresponding to the return electrodes.

However, in the related art, only a current generated in the photodiode of the pixel operating as the stimulation electrode is used, and thus, a magnitude of the current is small, such that accuracy is lowered.

### PRIOR ART DOCUMENT

### Patent Document

Korean Patent No. 10-1838150

### SUMMARY OF THE INVENTION

An object of the present disclosure is to propose a sub-retinal prosthesis using a multi-photodiode sensing technology capable of effectively delivering a stimulus and improving a spatial resolution.

According to an embodiment of the present disclosure, a sub-retinal prosthesis using a multi-photodiode sensing technology includes: a pixel array including a plurality of pixels of which k pixels are set as a unit group; and a digital controller controlling the pixel array to activate one of the k pixels as a stimulation electrode and activate the other k-1 pixels as return electrodes, wherein the unit group includes k photodiodes, a sensing circuit and a current driver, the sensing circuit outputs a stimulation parameter using currents each generated by the k photodiodes according to irradiation of light as an input, and the current driver outputs a stimulation current corresponding to the stimulation parameter.

Cathodes of the k photodiodes may be connected to each other.

The sensing circuits and the current drivers may be provided as many as the number of unit groups.

The k pixels may include a central pixel and a plurality of peripheral pixels adjacent to the central pixel.

The k pixels may include a central pixel and upper, lower, left, and right pixels adjacent to the central pixel.

The digital controller may activate one of the k pixels as the stimulation electrode, and activate the other k-1 pixels as the return electrodes after a preset time has elapsed.

The unit group may include a power supply voltage, a plurality of switches connected to the power supply voltage, and capacitors each connected to the plurality of switches and the k photodiodes.

### BRIEF DESCRIPTION OF THE DRAWINGS

FIGS. 1A and 1B are views for describing a current bleeding phenomenon in a retinal prosthesis according to the related art;

FIG. 2 is a diagram illustrating a layout of stimulation/return electrodes for removing a current bleeding phenomenon;

FIG. 3 is a view illustrating a configuration of a retinal prosthesis according to an embodiment of the present disclosure;

FIG. 4 is a view illustrating a unit group of the retinal prosthesis according to an embodiment of the present disclosure;

FIG. 5 is a view illustrating a detailed configuration of a pixel array according to the present embodiment;

FIG. 6 is a diagram illustrating a detailed structure of a unit group pixel according to an exemplary embodiment of the present disclosure;

FIG. 7 is a view illustrating a case where a stimulation electrode and return electrodes are simultaneously activated by way of example;

FIG. 8 is a view illustrating a method in which the stimulation electrode is first activated to inject a current into the retina, and the return electrodes are then activated after a predetermined delay time; and

FIG. 9 is graphs illustrating measurement results of photodiode currents when a photodiode of 20 pm x 40 pm and a photodiode of 80 pm x 40 pm are irradiated with light with an illuminance of 0 to 3900 lux.

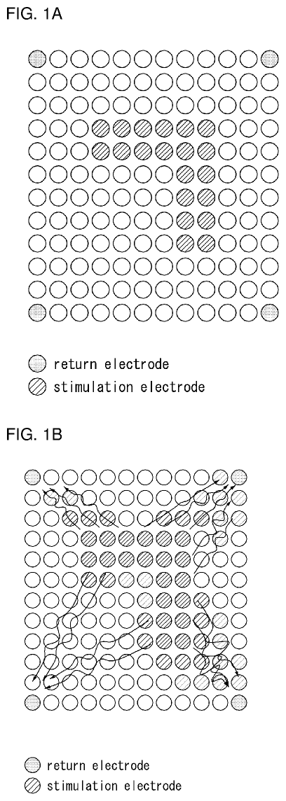

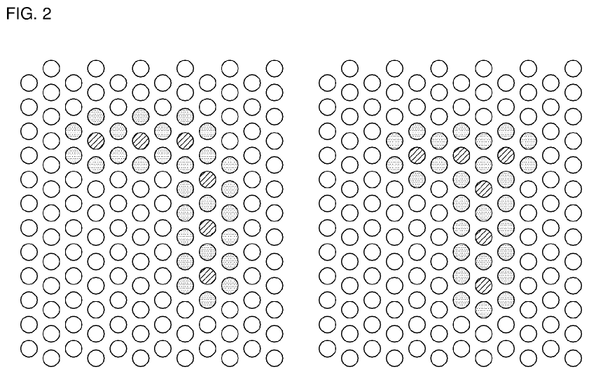

### DETAILED DESCRIPTION

The present disclosure may be variously modified and have several embodiments, and thus, specific embodiments will be illustrated in the drawings and be described in detail.

However, it is to be understood that the present disclosure is not limited to a specific embodiment, but includes all modifications, equivalents, and substitutions without departing from the scope and spirit of the present disclosure.

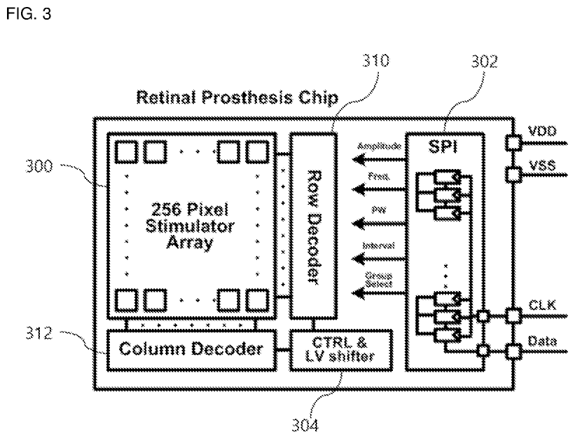

FIG. 3 is a view illustrating a configuration of a retinal prosthesis according to an embodiment of the present disclosure.

As illustrated in FIG. 3, the retinal prosthesis according to the present embodiment may include a pixel array 300, a serial-to-parallel interface (SPI) 302, and a digital controller 304.

The pixel array 300 includes a plurality of pixels of which k pixels are set as a unit group. The k pixels are sequentially activated as a stimulation electrode according to a preset order, and k-1 pixels other than the pixel activated as the stimulation electrodes are activated as return electrodes.

According to the present embodiment, sensing accuracy is increased by sharing photodiodes of a plurality of return electrodes adjacent to the stimulation electrode as well as a photodiode of the stimulation electrode.

That is, the present embodiment uses a multi-photodiode sensing technology.

The SPI 302 stores external data as parallel data, and a period, a pulse width, and the like, of a signal to be supplied to the pixel array 300 are determined by the parallel data.

The digital controller 304 controls the pixel array 300 to activate one pixel included in each group of the pixel array 300 as a stimulation electrode and activate the other pixels of each group of the pixel array 300 as return electrodes after a preset time elapses, that is, after a predetermined time delay, to detect a stimulation current.

As illustrated in FIG. 3, the digital controller 304 may detect a stimulation current of a pixel sensed by light through a row decoder 310 and a column decoder 312.

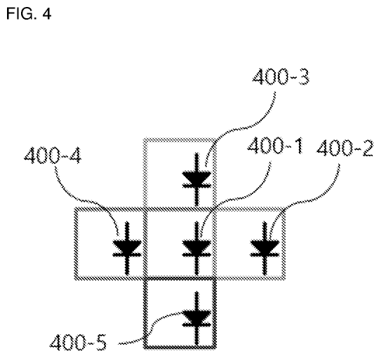

FIG. 4 is a view illustrating a unit group of the retinal prosthesis according to an embodiment of the present disclosure.

FIG. 4 illustrates a case where five pixels are set as one group, such that five photodiodes are shared.

Hereinafter, a case of sharing five photodiodes will be described by way of example, but the present disclosure is not limited thereto.

Referring to FIG. 4, a unit group includes five photodiodes 400-1 to 400-5, and outputs a stimulation current using currents each output from the five photodiodes when the five photodiodes are irradiated with light.

The multi-photodiode sensing technology according to the present embodiment increases an area of the photodiodes by connecting the photodiodes disposed in the plurality of pixels belonging to one group to each other.

When five photodiodes are included in one group, an area of the photodiodes increases five times.

Therefore, a photodiode sensing operation may be performed even with a small amount of light, such that a patient using the retinal prosthesis may secure a field of view even in a dark environment.

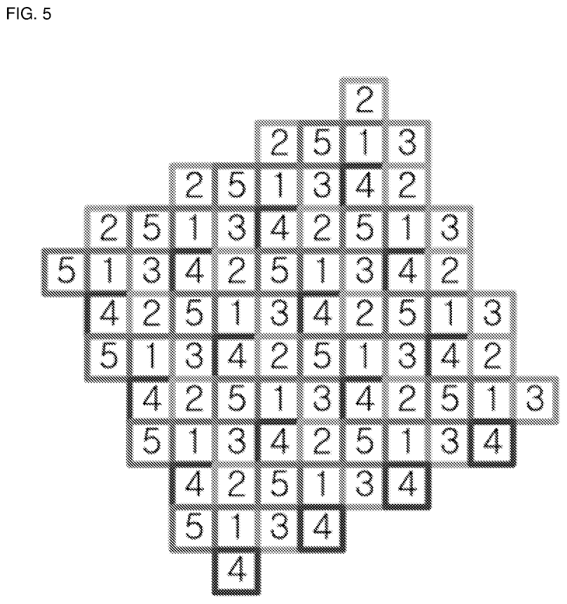

FIG. 5 is a view illustrating a detailed configuration of a pixel array according to the present embodiment.

Referring to FIG. 5, five pixels are set as one group, and reference numerals 1 to 5 refer to the order of the pixels activated as the stimulation electrode.

Each unit group includes a central pixel and upper, lower, left, and right pixels adjacent to the central pixel.

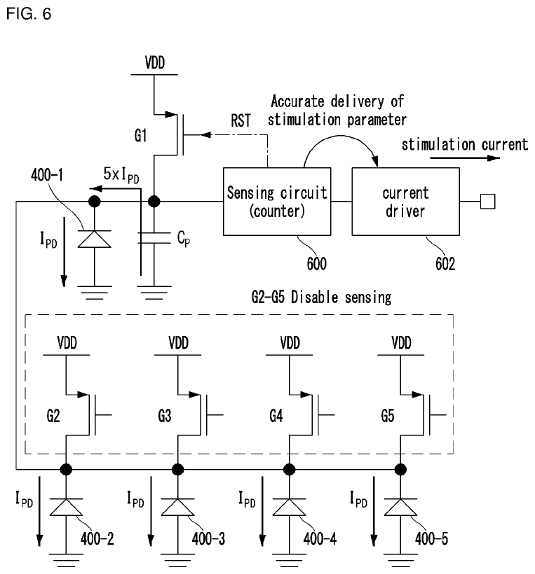

FIG. 6 is a diagram illustrating a detailed structure of a unit group pixel according to an exemplary embodiment of the present disclosure.

Referring to FIG. 6, a plurality of photodiodes 400-1 to 400-5 included in the unit group are connected to a power supply voltage VDD through a plurality of switches G1 to G5.

When the switches are turned on, cathodes of the photodiode 400-1 to 400-5 are charged with the power supply voltage.

Thereafter, when the photodiode are irradiated with light, voltages of the cathodes of the photodiodes decrease in proportion to an amount of light, and photodiode currents I_{PD} are generated to discharge parasitic capacitors CP of the photodiodes.

A sensing circuit 600 senses the photodiode currents to generate a stimulation parameter.

In more detail, when the parasitic capacitors are lowered to a predetermined value or less, a counter value of the sensing circuit 600 increases by 1.

The stimulation parameter generated as described above is supplied to a current driver 604, such that a stimulation current corresponding to the stimulation parameter is output.

According to the present embodiment, by connecting the cathodes of the photodiodes 400-1 to 400-5 of the five pixels to each other to configure a circuit, the sensing circuit 600 generates the stimulation parameter corresponding to the currents each generated by the five photodiodes. Therefore, the sensing circuit 600 may generate a counter value 5 times higher than a case of using a single photodiode, such that accuracy of light sensing may be further improved.

In a case of connecting the five photodiodes to each other as illustrated in FIG. 6, when the stimulation electrode and the return electrodes are simultaneously activated, there is a fear that current delivery may not be smooth due to narrow gaps between the stimulation electrode and the return electrodes.

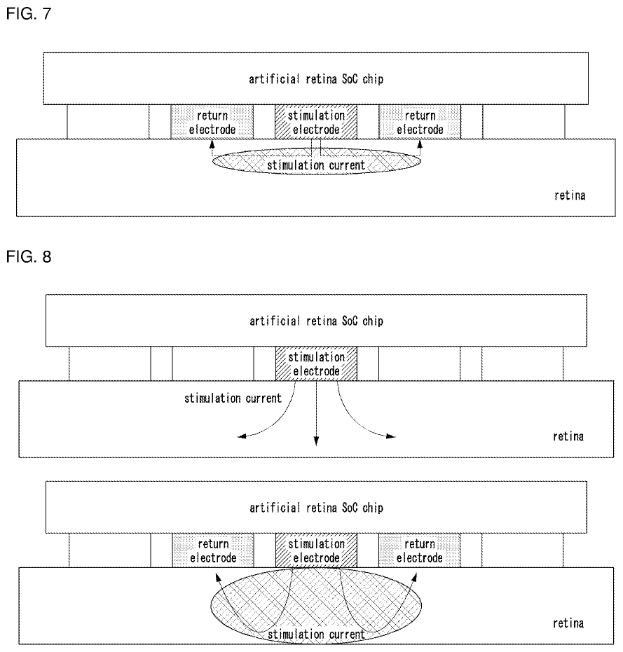

FIG. 7 is a view illustrating a case where a stimulation electrode and return electrodes are simultaneously activated by way of example.

In order to solve such a problem, a method in which the stimulation electrode is first activated to inject a current into the retina, and the return electrodes are then activated after a preset time (delay time) has elapsed as illustrated in FIG. 8 may be used.

Here, the delay time may be variously set from several ps to thousands of μs.

In addition, if an amount of return current is limited, the stimulation current relatively slowly returns through the return electrodes after the stimulation current spreads to the retina, such that a retinal stimulation effect may be improved.

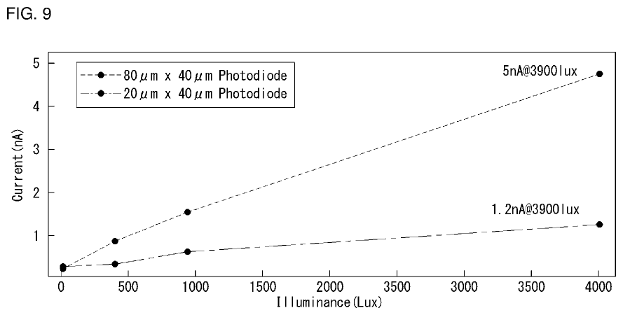

FIG. 9 is graphs illustrating measurement results of photodiode currents when a photodiode of 20 μm×40 μm and a photodiode of 80 μm×40 μm are irradiated with light with an illuminance of 0 to 3900 lux.

Photodiodes of a TSMC 180 nm general process were used. It can be seen that a photodiode current increases as a size of the photodiode increases and increases according to illuminance of the light with which the photodiode is irradiated.

According to the present embodiment, currents generated by all photodiodes included in the central pixel and a plurality of peripheral pixels adjacent to the central pixel are sensed, and thus, performance of the retinal prosthesis may be improved.

According to the present disclosure, it is possible to increase sensing accuracy by sharing the photodiodes disposed in the plurality of pixels adjacent to the pixel operating as the stimulation electrode to increase an area of the photodiodes.

In addition, according to the present disclosure, it is possible to minimize a current bleeding phenomenon even though the stimulation electrode and the return electrodes are disposed to be close to each other by applying a delay to activation times of the stimulation electrode and the return electrodes.

The above-described embodiments of the present disclosure have been disclosed for the purpose of illustration, various modifications, alterations, and additions may be made by those skilled in the art to which the present disclosure belongs without departing from the spirit and scope of the present disclosure, and it is to be considered that such modifications, alterations, and additions fall within the following claims.

What is claimed is:

1. A sub-retinal prosthesis using a multi-photodiode sensing technology, comprising:
a pixel array including a plurality of pixels of which k pixels are set as a unit group; and
a digital controller controlling the pixel array to activate one of the k pixels as a stimulation electrode and activate the other k-1 pixels as return electrodes, wherein the unit group includes k photodiodes, a sensing circuit and a current driver,
the sensing circuit outputs a stimulation parameter using currents each generated by the k photodiodes according to irradiation of light as an input, and
the current driver outputs a stimulation current corresponding to the stimulation parameter.

2. The retinal prosthesis of claim 1, wherein cathodes of the k photodiodes are connected to each other.

3. The retinal prosthesis of claim 1, wherein the sensing circuits and the current drivers are provided as many as the number of unit groups.

4. The retinal prosthesis of claim 1, wherein the k pixels include a central pixel and a plurality of peripheral pixels adjacent to the central pixel.

5. The retinal prosthesis of claim 1, wherein the k pixels include a central pixel and upper, lower, left, and right pixels adjacent to the central pixel.

6. The retinal prosthesis of claim 1, wherein the digital controller activates one of the k pixels as the stimulation electrode, and activates the other k-1 pixels as the return electrodes after a preset time has elapsed.

7. The retinal prosthesis of claim 1, wherein the unit group includes a power supply voltage, a plurality of switches connected to the power supply voltage, and capacitors each connected to the plurality of switches and the k photodiodes.
